(19) United States
(12) Patent Application Publication
(10) Pub. No.: US 2007/0207130 A1
(43) Pub. Date: Sep. 6, 2007

(54) Stem cell therapy to treat symptoms of avian flu and other diseases

(76) Inventor: Calvin Cao, Tampa, FL (US)

Correspondence Address: Lixian Jiang, 2102 Camp Indianhead Road, Land O Lakes, FL, 34639, US

(21) Appl. No.: 11/712,535

(22) Filed: Feb. 26, 2007

Related U.S. Application Data
(60) Provisional application No. 60/778,239, filed on Mar. 1, 2006.

(51) Int. Cl. A61K 35/12; C12N 5/08

(52) U.S. Cl. 424/93.7; 435/366

(57) ABSTRACT

The present disclosure relates to a method for the treatment of avian influenza using a stem cell preparation. Also described is a method for the manufacture of a stem cell preparation which can be used to treat the symptoms associated with avian influenza and a method for cryogenically preserving and/or storing a stem cell preparation which can be used to treat the symptoms associated with avian influenza. Also disclosed is a composition of matter containing stem cells which is useful for treating symptoms associated with avian influenza.

This application claims the benefit of U.S. Provisional Patent, Application No. 60/778,239, filed Mar. 1, 2006, which is incorporated herein by reference.

### FIELD OF THE INVENTION

The present invention relates generally to the field of cell biology of stem cells including human embryonic stem cells, cord blood stem cells as well as stem cells of other origin both human and non-human. More specifically, the present invention relates to a composition and method for the treatment of avian influenza and the symptoms associated therewith.

### BACKGROUND OF THE INVENTION

Worldwide influenza epidemics, also know as pandemics, have occurred in a random and unpredictable manner throughout history. Significant morbidity, mortality and economic loss has been attributed to each influenza pandemic. The influenza A virus can cause pandemics which occur as a result of mutations to the virus (also known as antigenic drift) which can lead to a virus sub-type to which no human immunity has developed. The lack of a human immune system response coupled with the ease of human-to-human transmission allows for localized outbreaks of influenza to spread globally.

The appearance of a mutated subtype of an influenza A virus can lead to a worldwide pandemic which will result in a high number of deaths, both in terms of actual numbers and percentage, significant morbidity and an extreme strain on the health care system not just in terms of the number of new patients but also in terms of health care worker illness. With the dramatic increase in globalization and urbanization occurring since the 1918-19 worldwide flu pandemic (which resulted in an estimated 50 million deaths worldwide), new pandemics caused by a new influenza virus are likely to spread very rapidly around the world. Of course, it is not possible to identify where or when the next flu pandemic will begin, but the risk is considered high enough that significant preparations are being taken worldwide.

Avian influenza is an infectious disease of birds caused by type A strains of the influenza virus. The disease occurs worldwide. While all birds are thought to be susceptible to infection with avian influenza viruses, many wild bird species carry these viruses with no apparent signs of harm. Other bird species, including domestic poultry, develop disease when infected with avian influenza viruses. In poultry, the viruses cause two distinctly different forms of disease—one common and mild, the other rare and highly lethal.

The second and far less common form of avian influenza, known as highly pathogenic avian influenza (HPAI), was first identified in Italy in 1878 and is characterized by sudden onset of severe disease, rapid contagion, and a mortality rate that can approach 100% within 48 hours. In this form of the disease, the virus not only affects the respiratory tract, as in the mild form, but also invades multiple organs and tissues with massive internal hemorrhaging.

To date, all outbreaks of HPAI have been caused by viruses of the H5 and H7 subtypes. Highly pathogenic viruses possess a tell-tale genetic “trade mark” or signature—a distinctive set of basic amino acids in the cleavage site of the HA—that distinguishes them from all other avian influenza viruses and is associated with their exceptional virulence.

Not all virus strains of the H5 and H7 subtypes are highly pathogenic, but most are thought to have the potential to become so. Recent research has shown that H5 and H7 viruses of low pathogenicity can, after circulation for sometimes short periods in a poultry population, mutate into highly pathogenic viruses. Considerable circumstantial evidence has long suggested that wild waterfowl introduce avian influenza viruses, in their low pathogenic form, to poultry flocks, but do not carry or directly spread highly pathogenic viruses. This role may, however, have changed very recently as some species of migratory waterfowl are now thought to be carrying the H5N1 virus in its highly pathogenic form and introducing it to new geographical areas located along their migratory flight patterns.

Apart from being highly contagious among poultry, avian influenza viruses are readily transmitted from farm to farm by the movement of live birds, people (especially when shoes and other clothing are contaminated), and contaminated vehicles, equipment, feed, and cages.

Influenza viruses are normally highly species-specific, and rarely infect other species. Since 1959, there has been only 10 documents cases of human infection with an avian influenza virus. However, influenza A has a segmented genome which increases the likelihood of antigenic drift thereby allowing influenza A viruses circulating in nature to display a high level of genetic variation and may develop the capability of crossing the species barrier. Of the hundreds of strains of avian influenza A viruses (16 H and 9 N recognized subtypes), only four are known to have caused human infections: H5N1, H7N3, H7N7, and H9N2. In general, human infection with these viruses has resulted in mild symptoms and very little severe illness, with one notable exception: the HPAI H5N1 virus.

Of all influenza viruses that circulate in birds, the H5N1 virus is the most significant threat to human health for at least two reasons. First, the H5N1 virus has caused by far the greatest number of human cases of very severe disease and the greatest number of deaths. It has crossed the species barrier to infect humans on at least three occasions in recent years: in Hong Kong in 1997 (18 cases with six deaths), in Hong Kong in 2003 (two cases with one death) and in the current outbreaks that began in December 2003 and were first recognized in January 2004.

A second implication for human health, of far greater concern, is the risk that the H5N1 virus—if given enough opportunities—will develop the characteristics it needs to start another influenza pandemic. The virus has met all prerequisites for the start of a pandemic except the ability to spread efficiently and sustainably among humans. While H5N1 is presently the virus of most immediate concern, the possibility that other avian influenza viruses, known to infect humans, might cause a pandemic cannot be ruled out.

All evidence to date indicates that close contact with dead or sick birds is the principal source of human infection with the H5N1 virus. Especially risky behaviors identified include the slaughtering, defeathering, butchering and preparation for consumption of infected birds. In a few cases, exposure to chicken feces when children played in an area frequented by free-ranging poultry is thought to have been the source of infection. Swimming in water which contains the carcasses of dead infected birds or which may have been contaminated by feces from infected ducks or other migratory birds is yet another suspected source of infection.

In many patients, the disease caused by the H5N1 virus follows an unusually aggressive clinical course, with rapid deterioration and high fatality. As is common to most emerging diseases, H5N1 influenza in humans is poorly understood. Initial symptoms have been reported to include a high fever, usually with a temperature higher than 38° C., and influenza-like symptoms. Diarrhea, vomiting, abdominal pain, chest pain, and bleeding from the nose and gums have also been reported as early symptoms in some patients. Watery diarrhea without blood appears to be more common in H5N1 avian influenza than in normal seasonal influenza. The spectrum of clinical symptoms may, however, be broader, and not all confirmed patients have presented with respiratory symptoms.

One feature seen in many patients is the development of manifestations in the lower respiratory tract early in the illness. Many patients have symptoms in the lower respiratory tract when they first seek treatment. Current evidence suggests difficulty in breathing develops around 5 days following the first symptoms. Respiratory distress, a hoarse voice, and a crackling sound when inhaling are commonly seen. Sputum production is variable and sometimes bloody. Limited data on patients in the current outbreak indicate the presence of a primary viral pneumonia in H5N1, usually without microbiological evidence of bacterial supra-infection at presentation. In patients infected with the H5N1 virus, clinical deteriorization is rapid, typically occurring in approximately 7 days. Another common feature is multiorgan dysfunction, notably involving the kidney and heart. Common laboratory abnormalities include lymphopenia, leukopenia, elevated aminotransferases, and mild-to-moderate thrombocytopenia with some instances of disseminated intravascular coagulation.

Limited evidence suggests that some antiviral drugs, notably oseltamivir (commercially available as Tamiflu), can reduce the duration of viral replication and improve prospects of survival, provided they are administered within 48 hours following symptom onset. However, prior to an outbreak in Turkey in late 2005 and early 2006, most patients had been diagnosed and treated late in the course of illness. For this reason, clinical data on the effectiveness of oseltamivir are limited. Moreover, oseltamivir and other antiviral drugs were developed for the treatment and prophylaxis of seasonal influenza, which is a less severe disease associated with less prolonged viral replication. Since its emergence in the late 1990's, more than half of the laboratory-confirmed cases of HPAI H5N1 have been fatal. H5N1 avian influenza in humans is still a rare disease, but a severe one that must be closely watched and studied, particularly because of the potential this virus to evolve in ways that could start a pandemic.

Stem cells are relatively undifferentiated cells of the same lineage (family type) that retain the ability to divide and cycle throughout postnatal life to provide cells that can become specialized and take the place of those that die or are lost. Stem cells represent a fundamental building block whose progeny eventually result in the estimated 220 types of specialized cells and tissues in a human. Stem cells are present in human embryos (in blastocysts), the placental complex, blood found in the umbilical cord at birth, bone marrow as well as numerous other tissues.

Recently the source of pluripotent hemopoietic stem cells has expanded to include autologous bone marrow, autologous and allogenic peripheral blood, cord blood and blastocysts. The term pluripotent is generally understood to describe stem cells that can give rise to cells derived from all three embryonic germ layers—the mesoderm, endoderm and ectoderm. Pluripotent stem cells are generally believed to have the ability to develop into essentially every cell, tissue and organ system found in the human body.

Currently stem cell suspension preparations are being used by Stem Cell Therapy International in Ukraine and Mexico to treat a wide variety of afflictions including cardiovascular disease, connective tissue disease, respiratory disease, digestive tract disorders, liver disease, kidney and urinary tract diseases, diabetes, diseases of the nervous system, the consequences of cerebral stroke, blood disorders, ocular disease as well as numerous other diseases, disorders and maladies.

Due to the significant threat posed by an H5N1 avian influenza pandemic coupled with the lack of a vaccine and the limited effectiveness of current therapies, the development of a stem cell based therapy to treat the symptoms associated with H5N1 avian influenza is highly desirable.

### SUMMARY OF THE INVENTION

In one embodiment, a method for the treatment of avian influenza using a stem cell preparation is described.

In an additional embodiment, a composition of matter which is useful for treating symptoms associated with avian influenza is described.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

All references, including publications, patent applications, and patents, cited herein are hereby incorporated by reference to the same extent as if each reference there individually and specifically indicated to be incorporated by reference and there set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the invention.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the claimed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it there individually recited herein.

As used herein “pharmaceutical composition” means a composition comprising a stem cell preparation and at least one ingredient that is not an active ingredient whereby the composition can be safely and effectively used as a product to obtain or achieve a desired outcome. The term “pharmaceutical composition” as used herein means compositions which result from the combination of individual components which are themselves pharmaceutically acceptable. For example, where intravenous administration is foreseen, the components are suitable or acceptable (in both quality and quantity) for intravenous administration. The stem cells of the present invention can be administered to mammals, namely humans and livestock, by numerous routes, such as intravenously, subcutaneously or intramuscularly. The dose administered may be between about 10,000 and about 5,000,000 cells per dose, about 250,000 to about 5,000,000 cells per dose or other amounts understood by a person of ordinary skill in the art to be therapeutically effective as a therapy to treat symptoms of avian influenza.

As used herein “daily basis” means in any given 24 hour period.

As used herein “daily amount” means the amount of the stem cell preparation that reaches the systemic circulation of the recipient of the stem cell therapy in any given 24 hour period from the pharmaceutical composition which has been administered.

As used herein “daily dose” means the total quantity of the pharmaceutical composition administered to the subject in any given 24 hour period independent of whether the quantity was administered in a single application or multiple applications during a 24 hour period.

The stem cell preparations are prepared and/or preserved according to Ukrainian patent application No. UA 60238 and Russian Federation patent application No. RU2233589 the disclosures of which are attached hereto and are hereby incorporated by reference in their entireties as if fully set forth herein.

The disclosure presented herein is directed towards a pharmaceutical composition which can be administered through a variety of routes including intravenously, intraosseously, subcutaneously, intramuscularly or directly into or onto the affected organ. When the pharmaceutical composition is delivered via an injection, the injection of the stem cell composition can occur as a single injection or multiple injections at any location inside or outside the body and the injection(s) can occur in a single day or over multiple days. The daily dose is administered to a subject wherein the daily amount of the stem cell preparation delivered to the subject from the pharmaceutical composition is about that which is therapeutically effective for treating symptoms associated with avian influenza. Additionally, the pharmaceutical composition may optionally include additional components such as salts, stabilizers and antimicrobials without departing from the spirit and scope of the claimed invention.

The stem cell composition useful for treating avian influenza can be administered at any time prior to the death of the subject from Avian influenza. For example, the stem cell composition could be administered as a prophylactic measure in a region where avian influenza has been detected; it can be administered after initial subject infection of the HPAI H5N1 virus and can be administered to the most vulnerable members of the population such as infants and the elderly.

The pharmaceutical composition of the present invention contains a stem cell preparation which has been previously defined and a pharmaceutically acceptable carrier. The quantity and nature of the stem cells to be incorporated in the composition will vary depending on desired therapeutic effect and the time span for which the composition is to provide a therapeutic effect. The quantity of stem cells in the pharmaceutical composition is that which will deliver a therapeutically effective amount for treating symptoms associated with avian influenza. Of course, the concentration and character of the stem cells to be included in the pharmaceutical composition will vary depending upon the components used in the composition, the route by which it is administered, the avian influenza symptom which requires treatment as well as other factors known to those of skill in the art.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed is:

1. A method for treating or preventing symptoms of avian influenza in a human subject comprising the steps of:
a. providing a composition comprising a plurality of stems cells; and
b. administering the composition to a human subject to treat of prevent symptoms associated with avian influenza.

2. The method of claim 1 wherein the composition comprises at least about 500,000 to over 5,000,000 stem cells.

3. The method of claim 2 wherein the composition comprises at least about 500,000 stem cells.

4. A pharmaceutical composition comprising:
a. a dose of stems cells; and
b. a pharmaceutically acceptable carrier,
wherein the pharmaceutical composition is suitable for treating or preventing symptoms of avian influenza in a human subject.

5. The pharmaceutical composition of claim 4 wherein the dose of stem cells comprises at least about 500,000 to over 5,000,000 stem cells.

6. The pharmaceutical composition of claim 5 wherein the dose of stem cells comprises at least about 500,000 stem cells.

7. The method of claim 1 wherein the composition comprises at least about 200,000 stem cell;

8. The method of claim 4 wherein the composition comprises at least about 200,000 stem cell;

9. The method of claim 1 wherein the composition comprises between about 200,000 to about 10,000,000 stem cell;

10. The method of claim 4 wherein the composition comprises between about 200,000 to about 10,000,000 stem cell;

11. The method of claim 1 wherein the composition comprises about 50,000,000 stem cell;

12. The method of claim 4 wherein the composition comprises about 50,000,000 stem cell;

13. The method of claim 1 wherein the composition comprises a combination of different types of cells;

14. The method of claim 4 wherein the composition comprises a combination of different types of cells;

15. The method of claim 1 wherein the composition is administered as a single dose or multiple doses for at least two days;

16. The method of claim 4 wherein the composition is administered as a single dose or multiple doses for at least two days;

17. The method of claim 1 wherein the composition is administered daily for about two weeks;

18. The method of claim 4 wherein the composition is administered daily for about two weeks;
